(19) United States
(12) Patent Application Publication
(10) Pub. No.: US 2014/0004783 A1
(43) Pub. Date: Jan. 2, 2014

(54) SELECTIVELY CHANGEABLE AIR FRESHENER

(71) Applicant: Air Raider Freshner, LLC, Southfield, MI (US)

(72) Inventors: Maurice Chandler, Southfield, MI (US); Gernard Mincey, Sterling Heights, MI (US)

(21) Appl. No.: 13/929,960

(22) Filed: Jun. 28, 2013

Related U.S. Application Data
(60) Provisional application No. 61/665,440, filed on Jun. 28, 2012.

(51) Int. Cl. A61L 9/12

(52) U.S. Cl. CPC A61L 9/125
USPC 454/156; 239/57

(57) ABSTRACT

This disclosure relates to an air freshener system. The system includes a housing providing an air flow path. The system is in communication with a plurality of scented objects. A selector is configured to move the scented objects from a stowed position into the air flow path. In one example, a control is operable to instruct the selector to move a particular one of the scented objects into the air flow path to provide air within the air flow path with a desired scent. In another example, the selector is manually operated.

### RELATED APPLICATIONS

This disclosure claims the benefit of U.S. Provisional Application No. 61/665,440 filed Jun. 28, 2012. The entirety of this provisional application is herein incorporated by reference.

### BACKGROUND

Vehicles, such as automobiles, have been known to include air fresheners to provide desired scents within the interior of the vehicle. One known type of air freshener clips to a vent within a vehicle cabin. Another known type of air freshener is incorporated into an air filter of an HVAC system. Buildings, such as homes and offices, also make use of air fresheners, such as plug-in air fresheners.

### SUMMARY

One aspect of this disclosure relates to an air freshener system. The system includes a housing providing an air flow path. The system further includes a plurality of scented objects. A selector is configured to move the scented objects into the air flow path. In one example, a control is operable to instruct the selector to move a particular one of the scented objects into the air flow path to provide air within the air flow path with a desired scent.

In another aspect of this disclosure, an air freshener system includes a housing providing an air flow path. The system further includes a plurality of scented objects, and a selector configured to move the scented objects from a stowed position into the air flow path. In this example, the selector is manually operable.

Yet another aspect of this disclosure relates to a method. The method includes establishing an air flow, and introducing one of a plurality of scented objects into the air flow to provide the air flow with a desired scent.

These and other features of the present disclosure can be best understood from the following drawings and detailed description.

### BRIEF DESCRIPTION OF THE DRAWINGS

The drawings can be briefly described as follows:

FIG. 1 schematically illustrates an example air freshening system including an automatic selection feature.

FIG. 2 schematically illustrates the interior of the disclosed air freshener housing.

FIG. 3 illustrates an example air freshener disc.

FIG. 4 schematically illustrates another example air freshening system including a manual selection feature.

FIG. 5 schematically illustrates the air freshening system according to this disclosure being used as a stand-alone unit.

FIG. 6 schematically illustrates the air freshening system according to this disclosure used in combination with an existing air conditioning unit.

### DETAILED DESCRIPTION

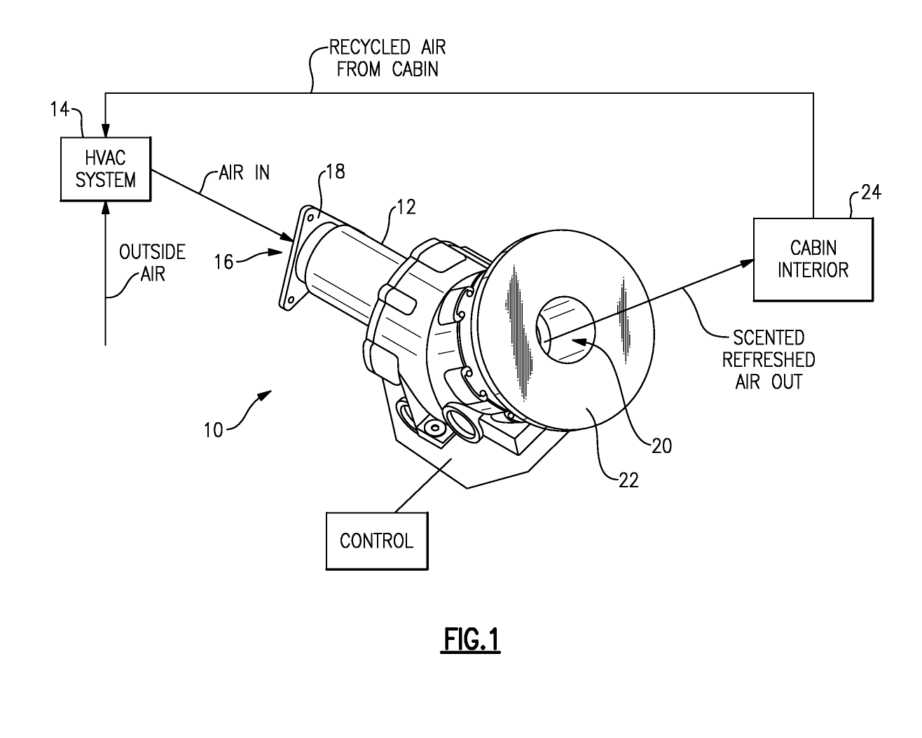

FIG. 1 illustrates one example of the disclosed air freshening system 10. In this example, the disclosed air freshening system 10 includes a housing 12 which is incorporated into an HVAC system 14. It should be understood that this disclosure extends to vehicles, including cars, airplanes and boats and further extends to buildings, such as homes or offices. While an HVAC system 14 is shown, this disclosure does not need to be associated with an HVAC system, as will be appreciated from the below.

With further reference to FIG. 1, the housing 12 includes an inlet orifice 16 provided adjacent an inlet flange 18, and an outlet orifice 20 provided adjacent and outlet flange 22. In this example, the housing 12 is mounted within an existing HVAC system 14 such that air from the HVAC system 14 travels into the inlet 16, through the interior of the housing 12, and out the outlet 20 toward a cabin interior 24, which is the space intended to be freshened by the scented air from the housing 12. Again, the cabin interior 24 could be any space, such as a vehicle interior or the interior of a building.

Notably, the housing 12 could be incorporated at any point within an HVAC system 14, depending on the intended application. For example, in the automotive context, the housing 12 could, in one example, be mounted in a location that is easily accessed for installation or for the replacement of scented discs. The HVAC system 14 could be sourced with either outside air, such as air from outside a vehicle, or recycled air from the cabin interior 24.

Further, it should be understood that while the exterior of the housing 12 is illustrated in a particular shape, that the shape of the housing 12 is not critical and can be changed as desired.

FIG. 2 schematically illustrates the detail of the interior of the housing 12. In the illustrated example, the housing 12 may include a fan 26, which is driven by a fan motor 28, and which is positioned within a main air flow path 30. As illustrated, a scented object, here an air freshener disc D1, is positioned downstream of the fan in the main airflow path 30. As the fan 26 draws air through the inlet 16, air passes over the disc D1 and is expelled from the air freshener housing 12 as scented, or refreshed, air.

Also within the housing 12 is a plurality of other air freshener discs, D2-D6. In this example, six discs D1-D6 are shown, however this example is not limited to any particular number of discs. Any number of scented objects can be incorporated into this disclosure. As illustrated, the discs D2-D6 are supported by a tray 32 which includes a number of slots 34 for supporting each of the plurality of discs. While discs D1-D6 are shown, this application extends to other scented objects that are not provided in a “disc” shape (e.g., the scented objects could be rectangular, triangular, etc.).

The housing 12 further incorporates a selector 36, which in one example includes an arm and a motor, which is capable of traveling axially within the housing (e.g., on a track) to selectively move the discs from a stowed position in the tray 32 into the air flow path 30, and to move the discs from the air flow path 30 back to the tray 32. In one example, the selector 36 moves one disc at a time, and operates similar to a CD changer. In this example, D1 is positioned in the main flow path, and the air flowing through the flow path 30 is scented depending on the scent associated with the disc D1.

The selector 36 is in communication with a control 38. The control 38 is a known type of computer, including a processor, memory, etc., and operable to instruct the selector 36 to move a particular one of the scented objects in response to a user input, as desired.

Discs D1-D6 are, in one example, scented differently from one another, and a user is able to select one of the discs D1-D6 depending on a desired scent for the air intended for the cabin interior 24. In another example, at least one of the discs D1-D6 is scented differently from at least one of the other discs. In yet another example, the discs D1-D6 have the same scent, and the user can select a new air freshener as the discs become depleted. It is further possible for a user to select more than one of the discs D1-D6 for entry into the flow path 30, thereby permitting a user to mix the scents of the discs together to provide air with a unique scent.

While the fan 26 is shown, the fan 26 is not necessarily required, as HVAC systems 14 typically provide air at a sufficient flow rate. That is, the air freshener housing 12 could be mounted at a point that takes advantage of an existing blower within the HVAC system 14, for example. The fan 26 can be used to accelerate an existing air flow, as desired by the user, via an instruction from the control 38 to the fan motor 28.

The fan 26 and motor 28 can, in some examples, be operable independent of operation of the HVAC system 14. In the case where the air freshening system 10 is used with an automobile, the fan motor could be powered by the automobile battery, and thus could operate independent of engine/HVAC 14 operation.

The control 38 can be incorporated into the cabin interior 24 such that a user can select, via some interface such as a touch-screen, between one or more scents (e.g., the scents associated with the discs D1-D6). The user further has control over operation of the fan 26. In one example where the vehicle is an automobile, the interface is a touch screen monitor available to the driver of the vehicle at the center console. In other examples, such as when the vehicle is a limousine, passengers that are separated from the driver are capable of having access to the interface. Further, while a control is illustrated in FIGS. 1-2, the control is not required in all embodiments. For example, the selector 36 can be a manual selector, such as that described relative to FIG. 4.

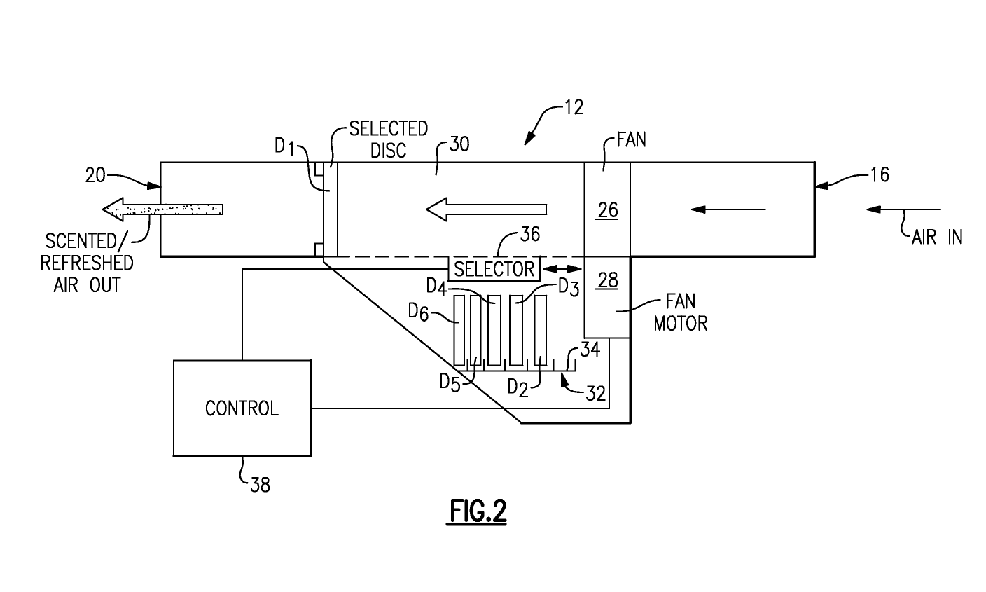

FIG. 3 shows an example air freshener disc, D1. In this example, the disc includes an outer ring portion 40 that is relatively rigid and capable of being grasped by the selector 36. The example disc D1 includes a highly concentrated scent portion 42 that is intended to be placed into the air flow path 30. The example air freshener disc D1 is ecofriendly, recyclable, and hypoallergenic. As explained above, the air freshener discs can be scented, and a user can select the types of scents to load into the housing 12.

It is possible that the housing 12 can be incorporated into an existing vehicle. That is, this disclosure is capable of being retrofit into an existing HVAC system 14. Accordingly, the details of the structure of the housing 12 are subject to change depending on the desired use of the air freshener system 10.

FIG. 4 illustrates another example of this disclosure. In this example, a plurality of discs D1, D2 are provided in separate, individual units 44, 46 that are attachable to a housing, or duct 48, providing an airflow passage 50. The units 44, 46, in this example, are coupled to the duct 48 by way of magnets 52. In this way, the units 44, 46 are selectively removable from the duct 48, which allows for ease in changing the units 44, 46 (and, in turn, the discs D1, D2). It should be understood that this disclosure extends to other examples where the units 44, 46 are attached to the duct 48 using other attachments, such as nuts and bolts. It should further be understood that more than one disc D1, D2 can be included in each unit 44, 46.

The units 44, 46 each include a manual selector. The manual selectors include a linkage 54 and a corresponding handle 56. The handle 56 is manually moveable, and movement of the handle 56 translates into movement of the linkage 54. The linkage 54 is attached to a corresponding disc D1, D2, and deploys the discs D1, D2 into the duct 48 in response to movement of the handle 56. In this example, the disc D1 is deployed, while the disc D2 is in a retracted position within the unit 46. A slit is formed in the duct 48 to allow the discs D1, D2 to be selectively moved in and out of the duct 48.

Similar to the above, as the discs D1, D2 are moved into the airflow passage 50 the airflow is freshened by way of the discs D1, D2. It should be understood that while only two units 44, 46 are illustrated in FIG. 4, that any number of discs can be placed along the air duct 48. Further, more than one disc can be deployed at one time, depending on the desired level of air freshening. Further, while linkages 54 are illustrated, it should be understood that any type of mechanism can be used to deploy the discs Dl. For example, a gearing system can be used. In fact, the units 44, 46 can be automated, as in the example of FIGS. 1 and 2.

FIG. 5 illustrates an example stand-alone system 58 according to this disclosure. The stand-alone system is particularly adapted for use independent of an existing HVAC system. For example, the stand-alone system may be placed on a surface 60, such as a floor or table top in a home or office. The example of FIG. 5 functions substantially similar to the examples of FIGS. 1-4, and may be manually or automatically operated, and thus those details are not repeated here for sake of brevity. A fan may be included in the system 58 of FIG. 5 example, again because the system 58 is separate from an existing HVAC system.

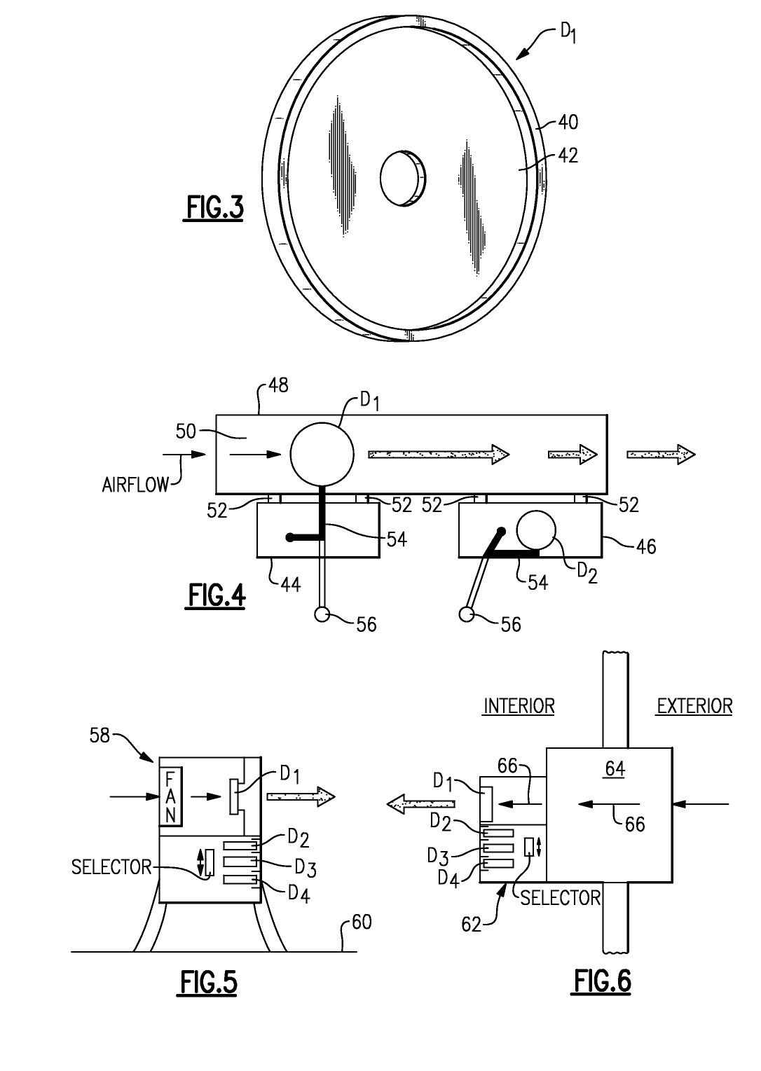

FIG. 6 illustrates an example where a system 62 is adapted for use with an air conditioning unit, such as a window air conditioning unit 64 commonly found in homes, offices, and apartments. In this example, the unit system 62 would be positioned at the interior of the space to be cooled (e.g., inside a window), and can selectively treat the airflow 66 from the air conditioning unit 64 in substantially the same manner as described above relative to FIGS. 1-4.

Among other things, this disclosure provides a system which allows a user to effectively control the type of scent delivered to a cabin interior. The disclosed air freshener further provides extended air freshener life, and avoids clutter within a cabin interior.

Although the different examples have the specific components shown in the illustrations, embodiments of this invention are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

What is claimed is:

1. An air freshener system comprising:
a housing providing an air flow path;
a plurality of scented objects;
a selector configured to move the scented objects into the air flow path; and
a control operable to instruct the selector to move a particular one of the scented objects into the air flow path to provide air within the air flow path with a desired scent.

2. The system of claim 1, wherein the scented objects are scented discs.

3. The system of claim 1, wherein the scented objects are stowed on a tray within the housing.

4. The system of claim 1, wherein at least one of the scented objects has a scent different from at least one of other of the scented objects.

5. The system of claim 1, wherein the housing is positioned within an HVAC system, and wherein the air flow path is configured to receive a flow of air from the HVAC system.

6. The system of claim 1, wherein the housing is a duct.

7. The system of claim 6, wherein the housing is a stand-alone housing, configured to be placed on a tabletop.

8. The system of claim 6, wherein the housing is configured to be attached to an air conditioning unit.

9. An air freshener system comprising:
a housing providing an air flow path;
a plurality of scented objects;
a selector configured to move the scented objects from a stowed position into the air flow path, the selector being manually operable.

10. The system of claim 9, wherein the selector is included within a removable unit attached to the housing.

11. The system of claim 10, including a plurality of selectors, each of the selectors included within an individual removable unit.

12. The system of claim 11, wherein the individual removable units are attached to the housing by way of magnets.

13. The system of claim 9, wherein the housing is a stand-alone housing, configured to be placed on a tabletop.

14. The system of claim 9, wherein the housing is configured to be attached to an air conditioning unit.

15. The system of claim 9, wherein the scented objects are scented discs.

16. The system of claim 9, wherein the scented objects are stowed on a tray within the housing.

17. The system of claim 9, wherein at least one of the scented objects has a scent different from at least one of other of the scented objects.

18. A method comprising the following steps:
establishing an air flow; and
introducing one of a plurality of scented objects into the air flow to provide the air flow with a desired scent.

19. The method of claim 18, wherein at least one of the scented objects has a scent different from at least one of other of the scented objects.

20. The method of claim 18, wherein the scented objects are scented discs.
